(19) United States
(12) Patent Application Publication
(10) Pub. No.: US 2015/0272145 A1
(43) Pub. Date: Oct. 1, 2015

(54) Method for Preparing Spoilage Resistant Raw Meat or Raw Seafood

(71) Applicant: Hill's Pet Nutrition, Inc., Topeka, KS (US)

(72) Inventor: Hungwei Lin, Lawrence, KS (US)

(21) Appl. No.: 14/660,971

(22) Filed: Mar. 18, 2015

Related U.S. Application Data
(60) Provisional application No. 61/971,489, filed on Mar. 27, 2014.

(51) Int. Cl. A23B 4/22; C12N 1/20; A23L 1/31; A23L 1/317; A23K 1/10; A23L 1/325

(52) U.S. Cl. CPC A23B 4/22; A23K 1/10; A23L 1/3252; A23L 1/3255; A23L 1/31; A23L 1/317; C12N 1/20; A23V 2002/00; A23Y 2240/00; A23Y 2280/00; A23Y 2260/00; A23Y 2220/00

(57) ABSTRACT

A method for preparing raw meat or raw seafood that is resistant to spoilage, comprising: a) incubating a solution comprising a population of non-pathogenic psychrotrophic lactic acid bacteria under conditions and for a period of time sufficient to enable growth of the bacterial population to form a bacterial culture capable of preventing spoilage; b) adding a saccharide to the bacterial culture and incubating the bacterial culture at a temperature of from 1 to 4° C., wherein the saccharide is present in an amount sufficient to achieve bacteriostasis of the bacterial culture; admixing the bacterial culture formed in step b) with the raw meat or raw seafood, wherein the bacteriostatic bacterial culture is present in an amount of from 1 to 20 weight % based on the total weight of the composition.

### BACKGROUND

Fresh ground meat or seafood blends are used extensively in pet food production, and the freshness of such material becomes very important for the quality, safety, and freshness of finished products. Even though refrigeration has been used to extend the freshness of fresh meat material, there is not always a guarantee that the refrigeration temperature will be consistently maintained below 40° F. (below 4.4° C.). Even so, there is a limited shelf life for the fresh raw material stored under refrigeration due to the enumeration of inherent psychrotrophic bacteria such as Pseudomonas species, Campylobacter species, Listeria species, etc.

Many factors may increase the risk to food safety and quality of using fresh meat/seafood material for food production, such as temperature infringement during transportation or storage, insufficient chilling of bulk material, extended storage beyond the shelf life, and cross contamination of pathogenic or spoilage microorganisms. As the industrial supplies of fresh meat slurries are packaged typically in a 2000-lb (907 kg) container (recognized as Combo) or 45,000-lb (20,411 kg) tank truck, maintaining low temperature of raw material is the most important method to keep raw material fresh and safe. The large size of bulk packaging makes the raw material located in the geometric center of the combo or tank truck most susceptible to temperature infringement. Such temperature infringements cause microbial spoilage and unexpected growth of pathogenic microorganisms. As mentioned earlier, many psychrotrophic microorganisms are able to enumerate under refrigeration conditions and undergo spoilage. Even though chemical antimycotics can be used to control or delay the microbial spoilage, excessive use of chemicals in foods is not popular among consumers or animals that eat the foods.

The use of lactic acid bacterial cultures in fermented or cured meat or seafood products, for example sausage, ham or fermented fish has previously been described (see for example U.S. Pat. No. 5,989,601, U.S. Pat. No. 6,884,455). The use of lactic acid cultures to preserve raw meat or seafood has also been described (see for example US2010/0310718 and GB 1,502,723). However, there is a need to improve the efficiency and effectiveness of such methods for retaining the freshness and preventing spoilage of raw meat or raw seafood.

Thus there is a need to provide new methods for preparing raw meat or raw seafood that is resistant to spoilage, in particular spoilage caused by pathogenic bacteria during temperature-infringed conditions. There is a particular need to provide such methods for bulk quantities of raw meat and seafood.

### BRIEF SUMMARY

The present invention aims to solve the above-identified problems. Specifically, the present invention provides method for preparing raw meat or raw seafood that is resistant to spoilage, the method comprising
a) incubating a solution comprising a population of non-pathogenic psychrotrophic lactic acid bacteria under conditions and for a period of time sufficient to enable growth of the bacterial population to form a bacterial culture capable of preventing spoilage of the raw meat or raw seafood;

b) adding a saccharide to the bacterial culture formed in step a) and incubating the bacterial culture at a temperature of from 1 to 4° C., wherein the saccharide is present in an amount sufficient to achieve bacteriostasis of the bacterial culture
c) admixing the bacterostatic bacterial culture formed in step b) with the raw meat or raw seafood, wherein the bacteriostatic bacterial culture is present in an amount of from 1 to 20 weight % based on the total weight of the composition, and is capable of preventing spoilage of the raw meat or seafood for at least 6 days at a temperature of from 5 to 9° C.

The current invention involves inoculating meat or seafood with adequate amounts of psychrotrophic microorganisms along with an energy source such as a saccharide as the fuel for promoting fermentation. Under unexpected temperature infringement, the psychrotrophic fermentation prevails in the meat or seafood slurry, which suppresses the outbreak of spoilage microorganisms so to retain the freshness of raw material upon an extended storage period. Under such uncertainties of raw material handling, fostering fermentation in the meat/seafood blend using lactic acid psychrotrophs establishes a healthy flora and excludes the growth of spoilage microbes in the raw material so to retain its freshness and secure its food safety. Step b) provides a combination of osmotic shock and cold shock conditions, leading to the bacteria achieving a bacteriostatic state, i.e. hibernation or dormancy of the psychrotrophic microorganisms. This provides the advantage that the use-fife of the fermentation inoculant is extended, and the presence of the saccharide energy source promotes quick growth of the microorganisms upon mixing with the raw meat or raw seafood, providing a more efficient and effective method for retaining the freshness and preventing spoilage of the raw meat or seafood.

In a further aspect, the present invention provides raw meat or raw seafood obtainable by the above-mentioned method. The present invention also provides a food composition comprising the raw meat or raw seafood. The food composition may be a pet food composition.

In an additional aspect, the present invention provides a fermentation inoculant comprising bacteriostatic non-pathogenic psychrotrophic lactic acid bacteria; and saccharide in an amount of at least 50 weight %.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present invention provides method for preparing raw meat or raw seafood that is resistant to spoilage, the method comprising
a) incubating a solution comprising a population of non-pathogenic psychrotrophic lactic acid bacteria under conditions and for a period of time sufficient to enable growth of the bacterial population to form a bacterial culture capable of preventing spoilage of the raw meat or raw seafood;
b) adding a saccharide to the bacterial culture formed in step a) and incubating the bacterial culture at a temperature of from 1 to 4° C., wherein the saccharide is present in an amount sufficient to achieve bacteriostasis of the bacterial culture;
c) admixing the bacterostatic bacterial culture formed in step b) with the raw meat or raw seafood, wherein the bacteriostatic bacterial culture is present in an amount of from 1 to 20 weight % based on the total weight of the composition, and is capable of preventing spoilage of the raw meat or seafood for at least 6 days at a temperature of from 5 to 9° C.

The raw meat or raw seafood is not especially limited. Examples are raw beef, pork, lamb, chicken, fish, prawn, duck, rabbit or venison, preferably beef. Typically the raw meat or seafood is ground or minced raw meat or raw seafood, preferably ground beef or ground chicken.

The term “resistant to spoilage” is intended to mean resistant to the growth of spoilage or pathogenic bacteria. Spoilage is the process in which food deteriorates to the point at which it is not edible by animals or its quality of edibility becomes reduced. The skilled person is aware of numerous methods for determining whether a food is spoiled.

The method of the invention typically prevents growth of spoilage psychrotrophic bacteria in the raw meat or raw seafood. Without being bound by theory, it is understood that the non-pathogenic psychrotrophic lactic acid bacteria prevent growth of spoilage bacteria by competing for nutrients with the spoilage bacteria and/or excreting metabolites that interfere with the growth of the spoilage bacteria. Such metabolites may be short chain acids, typically lactic acid.

Psychrotrophic bacteria are cold-tolerant bacteria that have the ability to grow at low temperatures, but have optimal and maximal growth temperatures above 15° C. and 20° C. respectively. Psychrotrophs are organisms that can grow at temperatures between about 0° C. to about 7° C. and produce visible colonies (or turbidity) within 7-10 days in this temperature range. Psychrotrophic bacteria are distinguished from psychrophilic bacteria which are cold-loving, having an optimal temperature for growth at about 15° C. or lower, a maximal temperature for growth at about 20° C., and a minimal temperature for growth at 0° C. The bacteria utilized in the present invention are non-pathogenic or non-spoilage psychrotrophic bacteria, preferably non-pathogenic psychrotrophic lactic acid bacteria. Examples of preferred non-pathogenic bacteria are Streptococcus, Enterococcus, Lactobacillus, Bifidobacterium, Pediococcus, Leuconotoc, and any combinations thereof.

It will be understood that the spoilage bacteria inherently present in the raw meat or seafood may also be psychrotrophic. The skilled person would have as part of their common general knowledge numerous psychrotrophic bacteria, including the non-pathogenic lactic acid bacteria added to the meat or seafood in accordance with the present, and the spoilage bacteria already present in the meat or seafood.

In step a) the solution comprises one or more nutrients sufficient to enable growth of the bacterial population. Typically, the one or more nutrients are selected from the group consisting of saccharides, a nitrogen source, fat, vitamins, minerals, micronutrients and combinations thereof. The nitrogen source may be selected from the group consisting of peptides, peptone, protein, hydrolyzed protein, nucleotides, yeast, yeast extract or whey. The saccharide is typically a mono or disaccharide. Preferably, the saccharide is selected from dextrose, sucrose, lactose, galactose, fructose, xylose, high fructose corn syrup and any combination thereof.

The minerals and micronutrients may be selected from the group consisting of sodium, potassium, calcium, magnesium, phosphate, chloride, and sulfur. Preferably, in step a) the solution comprises a saccharide in an amount of from 1 to 30 weight %. Preferably, the solution comprises whey and a saccharide. Most preferably, the saccharide is dextrose, also known as D-glucose. Such a solution is often termed a growth medium, and numerous typical bacterial growth media would be known to a skilled person.

Typically, prior to step a) a culture of the non-pathogenic psychrotrophic lactic acid bacteria is added to growth media in an amount of from 0.05 to 2 weight % to form the solution, preferably 0.1 weight %. Preferably the culture is a commercially available YO-Mix® 725 LYO 250 DCU culture (Danisco). This culture is understood to consist of Streptococcus thermophilus, Lactobacillus delbruecki, subsp. Bulgaricus, Lactobacillus acidophilus, Bifidobacterium lactis. The culture may alternatively be another commercially available yoghurt or sausage starter culture. Other sources for the bacterial culture are also contemplated.

The non-pathogenic psychrotrophic lactic acid bacteria are preferably selected from the group consisting of Streptococcus, Enterococcus, Lactobacillus, Bifidobacterium, Pediococcus, Leuconostoc, and any combinations thereof. Lactic acid bacteria convert carbohydrate to lactic acid plus carbon dioxide and other organic acids.

Most preferably, the non-pathogenic psychrotrophic lactic acid bacteria are selected from the group consisting of Streptococcus thermophilus, Lactobacillus delbrueckli subsp. bulgaricus, Lactobacillus acidophilus, Bifidobacterium lactis and combinations thereof. Other examples of lactic acid bacteria include Lactobacillus plantarum, Lactobacillus caret, Lactobacillus pentoaceticus, Lactobacillus brevis, Leuconostoc mesenteroides, Leuconostoc citrovorum, Leuconostoc dextranicum, Streptococcus lactis, Streptococcus cremis.

In step a) the solution is incubated at ambient conditions, typically a temperature of from 10° C. to 25° C., preferably about 15° C.

The growth of the bacterial population may be detected by a decrease in pH of the solution in step a). Usually, a pH of less than 4.0 demonstrates appropriate growth of the inoculant.

Preferably, in step a) incubation occurs until a total bacterial population of 10⁸ colony forming units per gram of solution is achieved. Usually, the period of time sufficient to enable growth of the bacterial population to form a bacterial culture capable of preventing spoilage of the raw meat or raw seafood is from 24 to 48 hours, most preferably about 48 hours.

The saccharide added in step b) is not especially limited provided it can be used to cause osmotic shock in the bacterial population. The saccharide is typically a mono or disaccharide. Preferably, the saccharide is selected from dextrose, sucrose, lactose, galactose, fructose, xylose, high fructose corn syrup and any combination thereof.

The amount of saccharide required to achieve osmotic shock and therefore bacteriostasis of the bacterial culture in step b) is usually at least 50 weight %, preferably at least 55 weight %. Most preferably, the saccharide is present in an amount of from 50 to 80 weight %, from 50 to 75 weight %, from 55 to 80 weight % or from 55 to 75 weight %. The skilled person would be able to determine the total saccharide concentration in the final inoculant required to achieve a bacteriostatic state without undue burden. An upper saccharide concentration limit of 75-80 weight % is likely due to the solubility of the saccharide in a water solution when it is stored under refrigeration e.g. a temperature of from 1 to 4° C. of temperature.

The combination of the presence of saccharide and refrigeration (typically at a temperature of from 1 to 4° C.) causes the bacteria to become bacteriostatic (which may also be described as dormant or a hibernation state). The bacteriostatic state is defined as a period of low metabolic activity in which the bacteria are unable to proliferate.

The bacteriostatic bacterial culture is added to the meat or seafood in an amount of from 1 to 20 weight % based on the total weight of the composition, preferably 2.5 to 5.0 weight %. The amount of 2.5 to 5.0 weight % inoculant is preferred since too little inoculant may not provide enough initial lactic acid bacteria to foster fermentation. Too high a concentration of the inoculant may dilute the nutrient content of the meat/seafood blend by carrying empty calories from sugar or fermented products like lactic acid, formic acid, acetic acid, propionic acid, butyric acid, which is not valuable for animal nutrition.

The prevention of spoilage may be measured by one or more of microbial count, change in pH or the identification of an odor compound. The odor compound may be selected from one or more of ammonia, hydrogen sulfide, methane thiol, trimethyl amine, methyl disulfide, dimethyl trisulfide, dimethyl sulfide, 2-methylbutanol, 3-methylbutanal, 3-methyl butanoic acid, 1-hexanol, pentanal, hexanal, heptanal, 1-penten-3-ol pentylfuran, benzaldehyde, acetic acid, propionic acid, pentanoic acid, hexanoic acid and heptanoic acid.

The odor compound may provide a putrefactive or spoilage odor. Examples of putrefactive or spoilage odors are ammonia, rotten/eggy/sulfur, rotten cabbage, fishy/putrid, green onion/sulfurous, or onion sulfurous odors.

Typically, spoilage of the raw meat or raw seafood is identified by a pH of from 6.5 or higher.

In a preferred embodiment the odor compound is identified by determining the quantity of odor compounds by gas-phase chromatography mass spectrometry. A GC-MS method for quantifying odor compounds produced by bacterial spoilage is described in Ercolini D et al, Appl Environ Microbiol, 2009 April; 75 (7): 1990-2001. The odor compounds may be volatile organic compounds. Some odor compounds are shown in Table 1 with their corresponding odors listed.

TABLE 1

| Compound | Odor Descriptor |
|---|---|
| Ammonia | Ammonia |
| Hydrogen sulfide | Rotten/eggy/sulfury |
| Methane thiol | Rotten cabbage |
| Trimethyl amine | Fishy/putrid |
| Methyl disulfide | Green onion/sulfurous |
| Dimethyl sulfide | Onion sulfurous |

The spoilage of the raw meat or raw seafood is prevented for a period of at least 6 days, preferably at least 8 days, most preferably up to 12 days at 6° C.

In a preferred embodiment, the total aerobic bacterial count of the raw meat or fish is less than or equal to 10⁵ after incubation with the bacterial culture for 8 days. In another preferred embodiment, the total aerobic bacterial count of the raw meat or fish is less than or equal to 10⁶ after incubation with the bacterial culture for 12 days. The total aerobic bacterial count may also be referred to as the total aerobic plate count, or simply total plate count. The method of obtaining the plate count is described in FDA Bacteriological Analytical Manual (or BAM method), Edition 8, Revision A, 1998. Chapter 3.

Typically, the spoilage psychrotrophic bacteria are selected from the group consisting of pseudomonas species, campylobacter species, and listeria species.

In a preferred embodiment the raw meat or raw seafood is from 500 kg to 30000 kg of raw meat or raw seafood. The present invention is particularly useful for bulk quantities of meat or seafood, because as mentioned above it is very difficult to avoid spoilage of bulk quantities by refrigeration alone, since it is difficult to maintain the geometric center of such large masses at the appropriate temperature. By carrying out thorough mixing of the inoculant with the meat or seafood, growth of the spoilage bacteria can be prevented throughout the bulk mass.

Preferably the raw meat or raw seafood is ground meat or ground seafood, or meat or seafood slurry, optionally ground beef or ground chicken.

In a typical embodiment, the raw meat or raw seafood is substantially free from chemical preservative. Examples of chemical preservatives typically used in meat blends are sodium nitrate, lactic acid, propionic acid, organic acid blend, sodium metabisulfite, or sodium bisulfate. The meat or seafood may be substantially free from antimycotic or antibiotic.

In a further aspect, provided is raw meat or raw seafood produced according to the method described above. The presence of the fermentation inoculant means that the raw meat or raw seafood is resistant to spoilage.

In an additional aspect, provided is a food composition comprising the raw meat or raw seafood as defined above. The food composition is typically a pet food composition. The pet food composition is not especially limited provided it comprises the raw meat or raw seafood of the invention. The pet food is typically a nutritionally complete pet food, for example one meeting the requirements of the Official Journal of the Association of American Feed Controls (AAFCO), 2013. The pet food is preferably suitable for a companion animal, most preferably a feline or canine animal. The pet food may be dry or wet pet food comprising the raw meat or seafood. The pet food may be a canned pet food.

In another aspect of the invention, provided is a fermentation inoculant comprising bacteriostatic non-pathogenic psychrotrophic lactic acid bacteria; and at least 50 weight % saccharide. Preferably the non-pathogenic psychrotrophic lactic acid bacteria are selected from the group consisting of Streptococcus, Enterococcus, Lactobacillus, Bifidobacterium, Pediococcus, Leuconotoc, and any combinations thereof. Most preferably the fermentation inoculant comprises Streptococcus thermophilus, Lactobacillus delbrueckli subsp. bulgaricus, Lactobacillus acidophilus, and Bifidobacterium lactis. The saccharide may be any saccharide or combination of saccharides as described above, preferably dextrose.

### EXAMPLES

### Example 1

A typical process of the invention is as follows:

Enumeration of a psychrotrophic culture is achieved by incubating the culture under ambient conditions for 48 hrs. Active psychrotrophs are preserved by incubation of the culture under high osmotic and refrigeration conditions. The culture is then blended with the meat or seafood blend in an amount of from 2.5 to 5 weight %, causing dispersion of the starter culture and energy source for fast fermentation of the psychrotrophs. The inoculated meat/seafood blend is then packaged into a 45,000 lb truck tank or a 2000 lb combo to improve food safety and increase use life by excluding growth of spoilage microorganisms.

### Example 2

Fresh ground beef was inoculated for psychrotrophic fermentation and was then stored for up to 12 days at 43±2° F. (6° C.). A yogurt starter culture (YSC) containing psychrotrophic lactic acid bacteria was purchased from a commercial supplier and grown in a sugar solution. The solution was sterilized by pasteurization at 208° F. (98° C.) tier 5 min as a growing media for SSC. After cooling, 0.1% of YSC was inoculated into the growing media then incubated under ambient conditions for 48 hrs to enumerate the YSC. After incubation, the growing media was mixed with equal amounts of dextrose powder to be used as a psychrotrophic fermentation inoculant (PFI). Fresh ground beef was purchased from a local grocery store and treated with 0%, 2.5% or 5% (w/w) of the PFI.

Apparent aroma, pH value, and microbial counts were measured to assess the freshness of the ground beef samples over up to 12 days. The pH and Total Plate Counts (TPC) of 0% PFI treated sample began to increase after 4 days of storage. In contrast, the pH in both treated samples significantly decreased from the initial level and the TPC were held steady during the first 8 days of storage. Typical spoilage and putrefactive odors were observed from the non-PFI treated sample after 6 days of storage while pleasantly sour and fermented smells were noticed from the 2.5% or 5% PFI treated samples throughout the 12-day storage period.

Methodology

Preparation of the PFI (Psychrotrophic Fermentation Inoculant)

A batch of 1 kg of starter culture solution was prepared as per the formula in Table 2. The nutrient solution was sterilized without the starter culture by heating the solution to 208° F. (98° C.) for 5 min. The solution was then cooled immediately to 100° F. (38° C.). Weight loss due to evaporation was compensated for using sterilized deionized water. The nutrient solution was packaged into a ½-gal (1.9 liter) Mason jar and then inoculated with 1 gm of YO-Mix® 725 LYO 250 DCU (Danisco). The inoculated solution was incubated at 72° F. (22° C.) for 48 hours. The pH of the solution was measured and monitored, with decreasing pH demonstrating proper growth of the starter culture. The starter culture blend was prepared by blending 1 kg of the starter culture solution with 1 kg of dextrose according to the recipe shown in Table 3. The PFI was packaged in quart-size Mason jars and stored in a refrigerator; the overall PFI formula is shown as Table 4.

Treatment of Fresh Ground Beef

Fresh ground beef (2.5 kg) was purchased from a local butchery and evenly divided for the following 3 treatments.

(1) Control: no treatment.
(2) Treated with 2.5% (w/w) of PFI.
(3) Treated with 5% (w/w) of PFI
The non-treated ground beef were blended alone using a Kitchen Aid Kitchen Mixer for 2 min. The treated samples were blended with either 2.5% or 5% (w/w) of PFI for 2 min. Each sample (90 gm) was packaged in an 11-cm petri dish then stored at a 43° F. refrigerator for up to 12 days. A sample of each treatment was removed from storage to deter nine its apparent organoleptic characteristics, pH value, total plate count of aerobic microorganisms, and total plate count of anaerobic microorganisms.

TABLE 2
Formula of starter culture solution

| Ingredient | % | Weight (gm) |
|---|---|---|
| Dextrose | 19 | 190 |
| Whey, dried | 1 | 10.0 |
| Water | 79.9 | 799.0 |
| YO-Mix ®725 Starter Culture | 0.1 | 1.0 |

TABLE 3
Formulation of starter culture blend

| Ingredient | % | Weight |
|---|---|---|
| Starter culture solution | 50 | 1 kg |
| Dextrose | 50 | 1 kg |

TABLE 4
Formulation of PFI Ingredient

| Ingredient | % |
|---|---|
| Dextrose | 59.5 |
| Whey, dried | 0.5 |
| YO-Mix ® 725 Starter Culture | 0.05 |
| Water | 39.95 |

Result and Discussion

The treated raw ground beef samples were observed with sweet, sour, fatty, with sausage like aromas over the 12 days of storage while the control non-treated sample had to be terminated on the 8^{th} day of storage due to its overwhelmed rotten and spoiled odors. Due to the considerations of meat spoilage potential to affect human health, the organoleptic observations were conducted by the experiment operators including the inventor instead of a sensory evaluation panel.

The pH value of non-treated fresh ground beef increased while that of the treated samples decreased over the storage period under refrigeration. The current study adopted a 43±2° F. (6° C.) temperature for storage to simulate a temperature infringed condition under refrigeration, which typically occurs during bulk storage, bulk transportation, or malfunction of handling equipment of the raw material.

TABLE 5
pH of ground beef with and without PFI

| | pH | | |
|---|---|---|---|
| Day | Control | 2.5% starter | 5% starter |
| 0 | 5.65 | 5.65 | 5.65 |
| 1 | 5.65 | 6.19 | 6.03 |
| 2 | 5.37 | 5.19 | 5.07 |
| 4 | 5.22 | 4.87 | 4.78 |
| 6 | 5.62 | 4.88 | 4.83 |
| 8 | 6.5 | 4.74 | 4.61 |
| 12 | | 4.62 | 4.66 |

As shown in Table 5, all samples started with an initial pH value of 5.6. The pH of the control sample decreased initially but started to creep up after 4 days of storage. As spoiled and deteriorating odors were observed in the control sample after 8 days of storage, the sample was terminated for the study. At that time, the pH of the control sample reached 6.5, after which point, as reported, beef begins to decompose. The pH of both PFI treated samples continued to decrease throughout the 12 days of storage period while no substantial and offensive odors were observed in either sample, but rather pleasantly sweet, sour and fermented smells were observed.

Examination of the microbial populations in the samples indicated both aerobic and anaerobic plate counts in the non-treated sample began to increase after 4 days of storage but were held steady in the treated samples for at least 8 days. Table 6 shows the total aerobic plate counts in different samples throughout the 12-day storage period.

TABLE 6
Total aerobic plate counts

| | Total aerobic plate counts (Log cfu) | | |
|---|---|---|---|
| Day | Control | 2.5% starter | 5% starter |
| 0 | 5.3 | 5.3 | 5.3 |
| 1 | 5.4 | 5.2 | 4.9 |
| 2 | 5.3 | 4.8 | 4.9 |
| 4 | 4.9 | 4.5 | 4.5 |
| 6 | 7.8 | 5.2 | 5.1 |
| 8 | 7.9 | 5.2 | 5.0 |
| 12 | | 6.3 | 6.7 |

Initially, all samples started with a 10⁵ level of aerobic plate counts and a 10⁴ level of anaerobic plate counts. The aerobic microbes were held out in all samples during the first 4 days of storage but increased afterward to near a 10⁸ level in the control sample. At such microbial level, meat is usually considered as spoiled. Along with the high pH of the control sample after 8 days of storage, it can be determined that the control sample was definitely spoiled after 8 days of storage. In contrast, the populations of the aerobic microbes in the treated samples were held steady at a 10⁵ level for at least 8 days then slightly increased to a 10⁶ level at the 12^{th} day of storage.

The populations of anaerobic microbes in all samples increased initially but were held steadily at a 10⁵ level during the first 4 days of storage as illustrated in Table 7.

TABLE 7
Total anaerobic plate counts

| | Total anaerobic plate counts (Log CFU) | | |
|---|---|---|---|
| Day | Control | 2.5% starter | 5% starter |
| 0 | 3.9 | 3.9 | 3.9 |
| 1 | 5.3 | 5.0 | 5.2 |
| 2 | 5.2 | 5.0 | 4.6 |
| 4 | 5.1 | 4.8 | 5.0 |
| 6 | 7.3 | 5.2 | 5.3 |
| 8 | 6.5 | 4.9 | 5.3 |
| 12 | | 5.9 | 6.7 |

In a similar pattern as observed previously, the anaerobes in the control sample began to increase after 4 days of storage while the treated samples were able to hold the growth of anaerobes for at least 8 days. These observations indicate that storing fresh ground beef under 43±2° F. (6° C.) without the psychrotrophic fermentation scheme may have up to 4 days of use life, after which microbes started to grow substantially and meat became decomposing. However, the PFI treated samples were able to delay such onset from 4 days to at least 8 days. It can be concluded that the idea of retaining the freshness of fresh meat/seafood blend using psychrotrophic fermentation of lactic acid bacteria or sausage starter culture is feasible.

### Example 3

### Delay Spoilage of Ground Raw Chicken at a Quality Near Spoilage Using Psychrotrophic Fermentation Treatment

The object of the experiment described below was to treat ground raw chicken near spoilage with 2.5% or 5% PFI with a view to delaying its spoilage even if the meat is stored at an elevated refrigeration temperature.

The impact of PFI treatment on organoleptic property, microorganism populations, pH, and volatile compounds of ground chicken near spoiled quality over storage time at 43±2° F. (6° C.) was determined.

Methodology

Sample Treatment

Commercial supply of fresh ground raw chicken was stored under typical refrigeration conditions for 5 days in a 2000 lbs capacity bulk container, when the total aerobic plate counts in the ground raw chicken reached a 10⁷ level before any excessive odor was perceived, at which stage quality of the meat was near spoiled but cannot be apparently detected using organoleptic evaluation.

The ground raw chicken (3 kg) was treated with the following 3 treatments as for the ground raw beef study:

(1) Control: with 3.25% (w/w) of sterilized deionized water.

(2) Treated with 2.5% (w/w) of PFI.

(3) Treated with 5% (w/w) of PFI

Each treatment with a total batch weight of 1 kg was blended with either deionized water or PFI using a Kitchen Aid Kitchen Mixer for 2 min. The blended mixture was divided into an 11-cm petri dish, with 90 gm of ground raw chicken for each sample, and then stored at a 43°±2° F. (6° C.) refrigerator for up to 12 days. A sample from each treatment was removed at 0, 1, 2, 5, 7, 9, and 12 days of storage to determine its apparent organoleptic characteristics, pH value, total plate counts of aerobic and anaerobic microorganisms, and intensity of volatile compounds.

Analysis of Volatile Compounds

Sample Preparation

Ground raw chicken samples were collected according to the previously mentioned method then stored inside a half-pint size Mason jar, flushed with nitrogen, sealed with the gasket ring, then stored in a freezer of 0° F. (−18° C.) temperature until required for analysis. The samples were thawed in a refrigerator for 2 hrs prior to the analysis. Each sample preparation was replicated once for statistical analysis purposes. Ground raw chicken sample (6 g) was weighed into a 600 mL glass beaker. Approximately 200 mL of liquid nitrogen was added to the ground chicken sample to freeze and harden the sample for grinding. The hardened ground raw chicken was transferred into a Waring blender. Sodium sulfate (6 gm) was added to the blender as a water binding agent. After mixing for 20 seconds, when the mixture formed granule like particles, an additional 9 gm of sodium sulfate along with 10 mL of liquid nitrogen was added to the blender. The mixture was further blended for another 30 seconds. The ground mixture was transferred to a ceramic mortar and pestle for fine grinding. Duplicates of the fine ground sample (4 g) were placed into a 20 mL glass vial, flushed with nitrogen, and sealed using a PTFE gasket and a metal cap for Solid Phase Micro Extraction gas chromatography mass spectrometry (SPME-GCMS) analysis.

SPME-GC-MS Analysis:

Extraction of volatile compounds from the ground chicken sample was performed using Solid Phase Micro Extraction (SPME) according to the following conditions:

SPME extraction fiber: 2 cm 50/30 um DVB/Carboxen/PDMS Fiber (Suppelco).

Volatile extraction and desorption protocol:

Fiber clean up: 250° C. for 6.0 min

Glass vial incubation temperature: 50° C.

Depth of fiber penetration inside the vial: 31 mm

Volatile extraction time: 60 min

Equilibration time: 30 min

Fiber penetration inside the GC injector: 67 mm

Desorption time: 5 min

GCMS Instrument Control Parameters:

### GC Oven Temperature Program

Soaking at 35° C. for 1 min then ramp at 6° C./min to 230° C.; soak at 230° C. for 10 min.
Run Time: 43.5 min
Post run: 10 min at 240° C.

### Front PTV Inlet

Carrier gas: He
Mode: Splitless
Pressure: 4.8678 psi
Gas flow rate: 53.83 mL/min
Septum purge flow rate: 3 mL/min
Purge flow to split Vent: 50 mL/min for 1 min.

### Column Carrier Gas Control

Pressure: 4.8678 psi
Flow rate: 0.83004 mL/min
Average Velocity: 32.952 cm/sec
Holdup Time: 1.5174 min
Flow Program: 0.83004 mL/min fir 43.5 min then purge at 1 mL/min for 10 min.

### Back SS Inlet

Carrier gas: He
Splitting mode: Split
Heater temperature: 250° C.
Pressure: 3.4051 psi
Total flow rate: 79.99 mL/min
Septum purge flow rate: 3 mL/min
Gas Saver: Off
Split Ratio: 50:1
Split Flow rate: 74.999 mL/min

### Auxiliary Thermal Device Control

Thermal Aux 1 (MSD Transfer Line): 250° C.
Thermal Aux 2 (User Configurable): 250° C.

### GC Chromatograph Columns:

Guard column #1: J&W 122-7332
Chromatograph coldrawingumn: DB-WAXetr, 260° C., 30 m×250 μm×0.25 μm
Post chromatograph column: J&W deactivated, 280° C. 2 m×200 μm×0 μm

### Mass-Spectrum Detector Parameters:

Tuning mode: automatic
Acquisition Mode: Scan
Vacuum pressure: 2.2×10⁻⁵ torr
Low mass ion set point: 34.0
High Mass set point: 403.0
MS source temperature control: 230° C. maximum 250° C.
MS quadrupole tube temperature control: 150° C. maximum 200° C.

Result and Discussion

Results of the current study demonstrate that the PFI treatment improves the freshness of ground raw chicken and delays the time of spoilage from less than 5 days to at least 12 days in four aspects which are: (1) reduction of pH, (2) reduction in odorous compounds, (3) reduction in lipid oxidation, and (4) increase in aroma compounds in the meat samples.

As for the results of the ground raw beef study, the organoleptic characteristics of the treated ground raw chicken samples on the 12^{th} day of storage included sweet, sour, fatty, with sausage like aromas, while the control, non-treated sample smelled rotten and spoiled. Additional observations were the formation of slimy and cloudy substances on the 5^{th} day of storage when the control, non-treated ground chicken study was ended due to its overwhelming and offensive odors.

In agreement with the previous results of the ground raw beef study, the pH of the treated ground raw chicken decreased significantly initially then held steadily over the 12-day storage time at 43±2° F. (6° C.) while the control, non-treated sample maintained its initial pH level. The initial pH value of the ground raw chicken meat (6.8) was high enough for the ground raw chicken to be considered spoiled as shown in Table 8.

TABLE 8
pH of ground chicken samples with and without PFI

| | pH of Meat blend under storage | | |
|---|---|---|---|
| Day | Control | 2.5% PFI | 5% PFI |
| 0 | 6.84 | 6.83 | 6.83 |
| 1 | 6.8 | 6.55 | 6.39 |
| 2 | 6.81 | 5.9 | 5.92 |
| 5 | 6.81 | 5.53 | 5.34 |
| 7 | — | 5.74 | 5.36 |
| 9 | — | 5.59 | 5.26 |
| 12 | — | 6.26 | 5.36 |

In general, meat is considered spoiled if its pH value exceeds 6.5. The pH values of the control, non-treated ground raw chicken maintained a pH of around 6.8 over the storage period up to 5 days, when the ground raw chicken was totally spoiled due to its overwhelming odors and slimy texture. In contrast, the pH values of both treated samples reduced initially after receiving the PFI treatment until reaching the lowest point at the 5^{th} day of storage, then the pH value of the 5% PR treated samples maintained a pH of around 5.4 throughout the 12 days of storage, while the 2.5% PFI treated sample maintained a pH value of around 5.6 for up to 9 days then began increasing. On the 12^{th} day of storage, the 5% PFI treated ground raw chicken still smelled fresh and sour with a slightly sweet and sausage aroma. As expected, the PFI treatment in the ground raw chicken sample fostered psychrotrophic fermentation by LAB to reduce the pH in ground chicken through acid production. The pH measurements demonstrate that the PFI treatment is effective in delaying meat spoilage, and that the 5% PFI treatment is more effective than the 2.5% PFI treatment in maintaining the low pH.

In addition to the high pH value, the near spoilage quality of the commercial supply of the ground raw chicken material can be indicated by its high initial total plate counts of both aerobic and anaerobic microorganisms of around 10⁷ CFU/gm. Meat with total plate counts exceeding this level is generally considered as spoiled,

TABLE 9
Total aerobic plate counts ground chicken

| | Total aerobic plate counts | | |
|---|---|---|---|
| Day | Control | 2.5% PFI | 5% PFI |
| 0 | 7.40E+07 | 6.50E+07 | 8.30E+07 |
| 1 | 4.60E+07 | 9.70E+07 | 6.90E+07 |
| 2 | 8.60E+07 | 8.90E+07 | 4.90E+07 |
| 5 | 4.30E+07 | 3.20E+07 | 2.90E+07 |
| 7 | | 5.10E+07 | 3.40E+07 |
| 9 | | 3.00E+07 | 2.60E+07 |
| 12 | | 5.00E+07 | 2.80E+07 |

TABLE 10
Total anaerobic plate counts ground chicken

| | Total anaerobic plate counts | | |
|---|---|---|---|
| Day | Control | 2.5% PFI | 5% PFI |
| 0 | 8.50E+07 | 6.30E+07 | 7.50E+07 |
| 1 | 6.40E+07 | 1.10E+08 | 9.50E+07 |
| 2 | 6.30E+07 | 8.00E+07 | 9.00E+07 |
| 5 | 8.40E+07 | 2.20E+07 | 5.50E+07 |
| 7 | | 3.70E+06 | 5.40E+07 |
| 9 | | 2.60E+07 | 1.80E+07 |
| 12 | | 3.10E+07 | 1.40E+07 |

According to Tables 9 and 10, all samples maintained around 10⁷ CFU/gm for both aerobic and anaerobic plate counts over the storage period except that the PFI treated samples show a slight decrease in their aerobic plate counts. The aerobic plate counts in the 5% treated ground raw chicken continued to decrease over the 12 days of storage period while that in the 2.5% treated sample decreased initially over first 5 days of storage then maintained around 4×10⁷ CFU/gm level in the later stage of storage.

The anaerobic plate counts of microorganisms in the PFI treated ground raw chicken increased from the initial level on the 1^{st} day of storage, which could be due to the inoculation of LAB from PFI. As shown in Table 10, the control, non-treated ground raw chicken maintained around the initial level of anaerobic plate counts over the 5 day storage period. On the 5^{th} day of storage, the control, non-treated sample had to be discarded due to its overwhelming rotten odor and slimy texture, which coincided with its high microorganism counts. After the initial spike, the anaerobic plate counts in both PFI treated samples steadily reduced over the remainder of the storage period.

The analyses of the volatile compounds in ground chicken samples clearly indicate the PFI treatments in the ground raw chicken reduce odorous compounds, increase sausage aroma, and delay lipid oxidation in the meat sample through the psychrotrophic fermentation of LAB. As sensory evaluation of the samples was not available in the current study due to health considerations, the analyses of volatile compounds become important to substantiate the results of the organoleptic observations. A total of 30 volatile compounds were identified in the current study using SPME-GCMS analysis as shown in Table 11.

TABLE 11

| | | | |
|---|---|---|---|
| | Retention | Retention | Target |
| Compound | Index | Time (min) | mass ion |

alcohols

| | | | |
|---|---|---|---|
| 1-Butanol | 1151 | 8.2 | 56 |
| 1-Penten-3-ol | 1166 | 8.47 | 57 |
| 1-Pentanol | 1257 | 11.15 | 42 |
| 1-Hexanol | 1362 | 12.82 | 56 |
| 1-Octen-3-ol | 1455 | 15.02 | 57 |
| Benzyl alcohol | 1889 | 23.33 | 108 |

aldehydes

| | | | |
|---|---|---|---|
| 2-methylbutanal | 915 | 3.81 | 57 |
| 3-methylbutanal | 920 | 3.87 | 58 |
| Pentanal | 982 | 4.87 | 44 |
| hexanal | 1087 | 6.83 | 56 |
| Heptanal | 1189 | 9.12 | 70 |
| (E)-2-Hexenal | 1223 | 9.8 | 41 |
| Octanal | 1293 | 11.5 | 84 |
| (E)-2-heptenal | 1334 | 12.3 | 83 |
| Nonanal | 1398 | 13.85 | 57 |
| (E)-2-Octenal | 1441 | 14.63 | 70 |
| Benzaldehyde | 1534 | 16.59 | 77 |
| (E)-2-Nonenal | 1548 | 16.88 | 70 |

esters

| | | | |
|---|---|---|---|
| Ethyl Butyrate | 1041 | 5.91 | 71 |

fatty acids

| | | | |
|---|---|---|---|
| Acetic acid | 1466 | 15.07 | 43 |
| Propanoic acid | 1553 | 16.8 | 74 |
| Butanoic acid | 1640 | 18.62 | 60 |
| 3-Methylbutanoic acid | 1678 | 19.45 | 60 |
| Hexanoic acid | 1858 | 22.74 | 60 |
| Heptanoic acid | 1966 | 24.6 | 60 |

other

| | | | |
|---|---|---|---|
| 2-Octanone | 1289 | 11.26 | 43 |
| Trimethylamine | 769 | 2.5 | 58 |
| 2-Pentylfuran | 1235 | 10.19 | 81 |
| methyl disulfide | 1079 | 6.66 | 94 |
| Dimethyl Trisulfide | 1389 | 13.6 | 126 |

Among those volatiles, we describe those volatile compounds with a clear trend of either an increase or a decrease in volatile intensity over the storage period among the different treatments in further detail below.

The odorous compounds detected from the ground raw chicken include trimethylamine, dimethyl trisulfide, dimethyl disulfide, 2-methylbutanal, 3-methylbutanal, 3-methyl butanoic acid, and 1-hexanol. As shown in Table 12, trimethylamine increased rapidly and significantly in the control, non-treated sample over the 5-day storage period.

TABLE 12
GC-MS Instrument response - Trimethylamine

| | GC-MS Instrument response - Trimethylamine | | |
|---|---|---|---|
| Day | Control | 2.5% PFI | 5.0% PFI |
| 0 | | 3389831 | 0 |
| 1 | 6608260 | 7924374 | 0 |
| 2 | 28551185 | 9123501 | 0 |
| 5 | 34155372 | 7047211 | 0 |
| 7 | | 8166692 | 0 |
| 9 | | 9468185 | 0 |
| 12 | | 22786070 | 0 |

Trimethylamine in the 2.5% PFI treated sample increased slightly during the first 2 days of storage, the level was maintained between 5 and 9 days of storage, then increased significantly on the 12^{th} day of storage. No trimethylamine was detected from any of the 5% PET treated samples in the current study. It is noted that the initial sample of the control, non-treated ground raw chicken was lost due to an unexpected instrument breakdown so its data is not included in the current report; however, the initial electronic intensity (EI) of GC-MS for each volatile compound discussed herein should be similar to that of the treated ground raw chicken samples. Trimethylamine has been used as an indicator for meat spoilage for the presence of biogenic amines due to its volatility for meat spoilage. As meat spoils, growth of bacteria degrades meat protein and forms biogenic amines, which are harmful to humans or animals if consumed. The results of the trimethylamine analysis indicate that the PFI treatment for the ground raw chicken has successfully delayed or inhibited the bacterial degradation of meat protein, and that the 5% PFI treatment is more effective than the 2.5% PFI treatment in inhibiting the formation of trimethylamine. Both PFI treatments are effective in delaying spoilage from less than 5 days to at least 12 days of storage time at 43° F. temperature conditions, even if the quality of starting material was near a spoiled state.

Both dimethyl trisulfide and dimethyl disulfide, are metabolites of bacteria in meat from the degradation of sulfur containing amino acids such as methionine or cysteine and have odor characteristics like strong garlic or ripe cheese.

TABLE 13
GC-MS Instrument response—Dimethyl Trisulfide
and Dimethyl disulfide

| | Dimethyl Trisulfide | | | Dimethyl disulfide | | |
|---|---|---|---|---|---|---|
| Day | Control | 2.5 | 5.0 | Control | 2.5 | 5.0 |
| 0 | | 0 | 0 | | 0 | 0 |
| 1 | 0 | 0 | 0 | 0 | 0 | 0 |
| 2 | 0 | 0 | 0 | 0 | 0 | 136949 |
| 5 | 601033 | 0 | 0 | 4436612 | 0 | 317930 |
| 7 | | 0 | 0 | | 274159 | 298419 |
| 9 | | 0 | 0 | | 262406 | 378232 |
| 12 | | 279282 | 130063 | | 945053 | 648183 |

As shown in Table 13, a significant level of dimethyl trisulfide was detected only on the 5^{th} day of storage in the control, non-treated ground chicken when the sample was observed as being very spoiled. By contrast, dimethyl trisulfide was detected at a significantly lower level, and only on the 12^{th} day of storage for PFI treated ground raw chicken. The EI level of dimethyl trisulfide found in the non-treated ground raw chicken is 2.2 and 4.6 times that found in the 2.5% and 5% PFI treated samples, respectively. Similarly, a significant level of dimethyl disulfide was also observed on the 5^{th} day of storage in the control, non-treated ground raw chicken when the sample was perceived as very spoiled, with only a slight increase for the treated samples. The EI level of dimethyl disulfide found on the 5^{th} day of storage in the non-treated sample is at least 4.7 times higher than that found in both PFI treated ground raw chicken on the 12^{th} day of storage. As both chemicals are reported to be associated with spoilage caused by the growth of Pseudomonas ssp. in meat, these results indicate that PFI treatment is effective in retaining the freshness or delaying the spoilage of ground raw meat, even if the starting raw material is near to a state of spoilage.

The 2- and 3-methylbutanal and methylbutanoic acid are typical bacterial associated volatiles through fermentation on the degraded amino acids.

TABLE 14
GC MS Instrument response

| | 3-Methylbutanal | | | Methyl butanoic acid | | | 2-Methylbutanal | | |
|---|---|---|---|---|---|---|---|---|---|
| Day | Control | 2.5 | Control | Control | 2.5 | 5.0 | Control | 2.5 | 5.0 |
| 0 | | 6157301 | | | 613823 | 851206 | | 3540393 | 3492623 |
| 1 | 6482609 | 5915965 | 3956512 | 779152 | 879363 | 0 | 3956512 | 3112864 | 3443998 |
| 2 | 7254845 | 5984915 | 4253028 | 1779533 | 2157772 | 0 | 4253028 | 3093993 | 3934766 |
| 5 | 11221384 | 7379865 | 6099732 | 9844286 | 4314215 | 0 | 6099732 | 3469680 | 2224262 |
| 7 | | 9238694 | | | 6432544 | 4334783 | | 4343428 | 2653037 |
| 9 | | 7984323 | | | 4755443 | 7395943 | | 4025578 | 3151863 |
| 12 | | 8408430 | | | 12346228 | 7366720 | | 4915703 | 4707203 |

The EI levels of 2- and 3-methylbutanal in the control, non-treated ground raw chicken increased slightly during the first 2 days then increased significantly on the 5^{th} day of storage (see Table 14). The EI levels of both volatiles were maintained around the initial level during the first 5 days of storage in the 2.5% PFI treated ground raw chicken, with a slight increase thereafter. The 5% PFI treated ground raw chicken was able to maintain its initial level of both volatiles with only a slight increase on the last (12^{th}) day of storage.

The final EI levels of both volatiles in both treated ground raw chicken samples were lower than that in the control, non-treated sample stored only for the 5 days by at least 10%.

All samples had a low initial level of methylbutanoic acid (see Table 14). The control, non-treated ground raw chicken had an approximately 12 fold increase in the level of methylbutanoic acid by the 5^{th} day of storage from the initial level, while it took the 2.5% PFI treated sample 12 days to reach this level. Except for the initial low amounts of methylbutanoic acid detected in the 5% PFI treated ground raw chicken, the volatile compound was not detected again in the same sample until the 7^{th} day in the storage. The final EI level of methylbutanoic acid in the 5% PFI treated ground raw chicken was significantly lower than that detected from the 5^{th} day of the control, non-treated sample or from the 12^{th} day of the 2.5% PR treated ground raw chicken by at least 25%. The results for these three volatile compounds containing a branched chain methyl group suggest both PFI treatments are effective in retaining the freshness or delaying the spoilage of ground raw meat, with the 5% PFI treatment being more effective than the 2.5% PFI treatment.

1-Hexanol was found to increase in the control, non-treated ground raw chicken at a faster rate than in both PFI treated samples. 1-Hexanaol has a wine, fatty, and fruity aroma perception and is associated with meat spoilage.

TABLE 15

| | GC MS Instrument response | | |
|---|---|---|---|
| | 1-Hexanol | | |
| Day | Control | 2.5 | 5.0 |
| 0 | | 16493231 | 20584844 |
| 1 | 30097886 | 20046796 | 25946080 |
| 2 | 42021764 | 26436097 | 31808186 |
| 5 | 57857623 | 28711862 | 19920477 |
| 7 | | 39716682 | 39872739 |
| 9 | | 16240478 | 45049491 |
| 12 | | 23786683 | 64087606 |

According to Table 15, on the 5^{th} day of storage, the 1-hexanol concentration in the control, non-treated ground chicken was at least 2 times higher than that in the treated samples. It took about 12 days for the 5% PFI treated ground raw chicken to reach a similar level of 1-hexanol as that detected from the control, non-treated ground chicken stored only for 5 days at 43° F. It is unknown why the 2.5% PFI treated raw chicken generated lower 1-hexanol during the 9^{th} and 12^{th} days of storage than the 5% PFI treatment. 1-Hexanol was reported for its association with meat spoilage. Measurement of 1-hexanol levels suggests that PFI treatment in ground raw meat is effective in delaying the meat spoilage.

The PFI treatment helps to retard oxidation in the ground raw chicken as indicated by measurement of key volatile compounds derived from lipid oxidation in the ground raw chicken samples. Those key lipid oxidation compounds detected in the current study include pentanal, hexanal, heptanal, and 1-penten-3-ol, among which hexanal is typically used as a key indicator for lipid oxidation. Other lipid oxidation compounds are also present in the ground chicken such as heptenal, hexenal, octanal, hexenal, and nonanal but their EI levels did not show a clear trend. As shown in Table 16, the control, non-treated sample maintained the highest EI level of pentanal throughout the 5 days of storage while both treated samples had a slight reduction in the pentanal levels over the 12-day storage period.

The levels of pentanal in the treated samples on the 12^{th} day of storage were at least 22% lower than that detected in the control, non-treated ground raw chicken stored for 5 days. The levels of hexanal showed a similar pattern to pentanal among the ground raw chicken samples as illustrated in Table 16.

TABLE 16
GC MS Instrument response

| | Pentanal | | | Hexanal | | |
|---|---|---|---|---|---|---|
| Day | Control | 2.5 | 5.0 | Control | 2.5 | 5.0 |
| 0 | | 118125527 | 103326799 | | 336534625 | 302549355 |
| 1 | 132699223 | 103222930 | 95008975 | 369919763 | 308513118 | 298855299 |
| 2 | 127721103 | 96081284 | 88033283 | 355439663 | 318481929 | 302322388 |
| 5 | 133040072 | 92384977 | 71129208 | 357044728 | 307096902 | 260016914 |
| 7 | | 98806213 | 72599306 | | 304794074 | 266298772 |
| 9 | | 82101398 | 73816555 | | 258818258 | 261703377 |
| 12 | | 84292230 | 75720226 | | 258518604 | 252522647 |

The control, non-treated sample had a rapid increase in the EI levels of heptanal over the 5 days of storage while the treated samples maintained their initial levels as shown in Table 17. The heptanal level in the control, non-treated ground raw chicken stored only for 5 day was at least 1.8 times greater than that in the treated samples stored for 12 days under the same conditions. These 3 aldehydes are typical breakdown products through oxidation of linoleic acid. These results illustrate that the PFI treatment is effective in retarding the lipid oxidation.

TABLE 17

| | GC MS Instrument response | | |
|---|---|---|---|
| | Heptanal | | |
| Day | Control | 2.5 | 5.0 |
| 0 | | 24179335 | 29162688 |
| 1 | 35367978 | 29417930 | 30597977 |
| 2 | 42884038 | 32996804 | 32685205 |
| 5 | 66153581 | 34200778 | 30647824 |
| 7 | | 36271484 | 32509547 |
| 9 | | 31563008 | 32183599 |
| 12 | | 35798596 | 33196622 |

1-Penten-3-ol is a breakdown product formed by oxidation of n-3 highly unsaturated fatty acids like linolenic acid (C18:3n3) and eicosapentanoic acid (C20:5n3) in ground raw chicken. This compound has been used for monitoring the oxidation of highly unsaturated n-3 fatty acids such as fish oil or products containing fish oil.

TABLE 18
GC MS Instrument response

| | 1-Penten-3-ol | | | pentylfuran | | | benzaldehyde | | |
|---|---|---|---|---|---|---|---|---|---|
| Day | Control | 2.5 | 5.0 | Control | 2.5 | 5.0 | Control | 2.5 | 5.0 |
| 0 | | 24220419 | 22356796 | | 4452764 | 13310089 | | 1388238 | 3961436 |
| 1 | 20003198 | 19802675 | 21476441 | 7241745 | 6858626 | 18039556 | 2727149 | 2099926 | 6849337 |
| 2 | 20805072 | 19385579 | 20165236 | 8573752 | 15176152 | 35246668 | 4157201 | 5415470 | 14757672 |
| 5 | 21130666 | 19013663 | 10295644 | 10588689 | 26047816 | 26189482 | 5621996 | 9501455 | 4378619 |
| 7 | | 19428341 | 12048283 | | 29610337 | 35777110 | | 15804991 | 8920752 |
| 9 | | 10116035 | 11415289 | | 8959625 | 39687686 | | 3839959 | 15429860 |
| 12 | | 9456213 | 14054904 | | 10960266 | 37735502 | | 4783390 | 23000679 |

As shown in Table 18, the control, non-treated ground chicken maintained the initial EI level of 1-penten-3-ol over the 5 day storage period. The EI level of 1-penten-3-ol in the 2.5% PFI treated ground raw chicken reduced slightly on the 1^{st} day of storage, held steadily around that level between the 2^{nd} and 7^{1h} days of storage, and then decreased again during the later days of storage. In a similar pattern, the EI levels in the 5% PFI treated ground raw chicken decreased significantly, by 49%, on the 5^{th} day of storage from the initial level, then maintained around that level with only a slight increase on the 12^{th} day of storage. These volatile compounds formed by lipid oxidation may be present in the ground chicken in a dynamic system, as existing volatiles may escape to the environment while new volatile compounds are generated. The gain or loss of such volatile compounds depends on the rate of lipid oxidation. The decrease in levels of these key lipid oxidation compounds such as pentanal, hexanal, heptanal, and 1-penten-3-ol in the treated ground raw chicken demonstrates that the PFI treatment is effective in lowering the lipid oxidation rate in the ground chicken in comparison to the control, non-treated sample. These observations of lipid oxidation compounds in the ground raw chicken, including maintaining a lower EI level of pentanal, hexanal, heptanal, and 1-penten-3-ol in the PFI treated ground raw chicken versus the control, non-treated sample demonstrate that the PFI treatment is effective in retarding or delaying lipid oxidation.

The formation of good aromas by the PFI treatment can be illustrated by pentylfuran and benzaldehyde, which are frequently found in dry fermented sausage products like pepperoni or salami. Even though these compounds are also derived from lipid oxidation, they are formed through enzymatic lipolysis and oxidative degradation of the liberated fatty acids. Pentylfuran is a degraded product form linoleic acid but reported frequently from cured sausage products to denote a meat fermentation aroma. As shown in Table 18, the pentylfuran increased only slightly in the control, non-treated ground chicken but increased rapidly and significantly in the 5% PFI treated samples during the first 2 days of storage, then maintained around that high level throughout the rest of storage period. The level of pentylfuran increased gradually in the 2.5% PFI treated ground raw chicken during the first 7 days of storage then decreased significantly afterward.

Benzaldehyde has an almond like aroma and is found in many sausage or ham products. As shown in Table 18, the 5% PFI treated ground raw chicken had the highest level of benzaldehyde followed by the 2.5% PFI treatment. Only a slight increase in benzaldehyde was observed in the control, non-treated ground raw chicken over the 5-day storage period. Benzaldehyde continued to increase in the 5% PFI treated ground raw chicken and peaked at the 7^{th} day of storage in the 2.5% PFI treated sample then began decreasing. These observations demonstrate that the PFI treatment is able to effectively generate desirable meat fermentation aroma compounds like pentylfuran and benzaldehyde through psychrotrophic fermentation, with the 5% PFI treatment being more effective than the 2.5% treatment.

Straight chain volatile fatty acids such acetic, propionic, butanoic, pentanoic, hexanoic; and heptanoic acid were observed in the ground raw chicken samples over the storage time; however, the PFI treatment shifted significantly the profile of the presence of these acids. Tables 19 and 20 illustrate the EI levels of straight chain volatile fatty acids. The control, non-treated ground raw chicken had higher levels of propionic acid and butanoic acid than the treated samples, while the treated samples had a higher proportion of the other acids. It is not known whether these volatile fatty acids were formed through psychrotrophic fermentation, microorganism metabolism, break down from lipid oxidation, or oxidation from other microorganism metabolites like alcohols or ketones.

TABLE 19
GC MS Instrument response

| | acetic acid | | | propionic acid | | | butanoic acid | | |
|---|---|---|---|---|---|---|---|---|---|
| Day | Control | 2.5 | 5.0 | Control | 2.5 | 5.0 | Control | 2.5 | 5.0 |
| 0 | | 1573229 | 5162840 | | 378554 | 473061 | | 1201119 | 1242934 |
| 1 | 3512609 | 2982312 | 6551415 | 830714 | 826591 | 695359 | 2466476 | 4116561 | 2644109 |
| 2 | 5362094 | 3535260 | 12091163 | 3742854 | 2028956 | 1346040 | 6074515 | 7995680 | 5007861 |
| 5 | 5359087 | 4224743 | 8421482 | 15805088 | 3191430 | 2794211 | 44783389 | 11666283 | 9964331 |
| 7 | | 4656991 | 8686469 | | 3802640 | 2861933 | | 12681084 | 9797816 |
| 9 | | 1836265 | 8829544 | | 3967206 | 2608888 | | 11630156 | 10129486 |
| 12 | | 1939727 | 9360450 | | 5496714 | 3058940 | | 6018416 | 9788452 |

TABLE 20
GC MS Instrument response

| | hexanoic acid | | | heptanoic acid | | | Ethyl butyrate | | |
|---|---|---|---|---|---|---|---|---|---|
| Day | Control | 2.5 | 5.0 | Control | 2.5 | 5.0 | Control | 2.5 | 5.0 |
| 0 | | 8339664 | 13533962 | | 204971 | 0 | | 0 | 0 |
| 1 | 24887232 | 13815702 | 24973449 | 382526 | 396821 | 0 | 0 | 0 | 0 |
| 2 | 32428938 | 44429283 | 66930306 | 408382 | 411813 | 587242 | 0 | 0 | 231660 |
| 5 | 34489141 | 76911823 | 79699952 | 554379 | 560969 | 644761 | 2017847 | 405184 | 0 |
| 7 | | 87816913 | 95680804 | | 808029 | 767788 | | 1474592 | 155781 |
| 9 | | 58974262 | 106514111 | | 625727 | 961256 | | 0 | 260577 |
| 12 | | 59173351 | 83471273 | | 499933 | 658950 | | 0 | 570325 |

The 5% PFI treated ground raw chicken had the highest production of acetic acid while the 2.5% PFI treated sample had the lowest production. The peak level of propionic and butanoic acids in the control, non-treated ground chicken were at least 3.5 times higher than that in the PFI treated samples. The levels of both propionic and butanoic acids were found to increase slowly but steadily in the PFI treated samples during the first 5 days of storage then maintained a low level over the rest of storage period. In contrast, both PFI treated ground raw chicken had higher production of hexanoic and heptanoic acids than the control, non-treated sample. Butanoic acid has been reported from meat spoilage and had a pungent and sour odor while hexanoic, and heptanoic acid are only observed in processed or cured meat products. It is clear that the psychrotrophic fermentation fostered by PFI treatment has successfully changed the microorganism profile in the ground raw chicken and shifted the production of their metabolites.

In summary, analysis of volatile compounds in ground chicken evidences that fostering psychrotophic fermentation in commercial meat or seafood blends at a quality near spoilage using the PFI treatment has improved the freshness and delayed the spoilage of the meat through reduction in the production of odor compounds such as trimethylamine, dimethyl sulfide, methyl butanoic acid, butanoic acid, 2-methyl butanal, and 3-methyl butanal, through increase in production of desirable aroma such as pentylfuran, and through delaying lipid oxidation such as decrease in the formation of pentanal, hexanal, heptanal, and 1-penten-3-ol.

The present invention can be applied in many areas for delaying spoilage or improving the freshness and safety of commercial meat or seafood blend in bulk packaging. For example, commercial meat supply for making canned or extruded pet food products are usually transported in bulk containers and subjected to days of storage under refrigeration. Treating the meat or seafood blend using PFI will not only help to retain the freshness and delay the spoilage of meat or seafood blend but also reduce or minimize the production of toxic compounds such as biogenic amines and microtoxins. In a typical protein rendering facility for processing dry meat or seafood meal, incoming material is usually stored in a large pool or pit awaiting rendering. Treating the incoming raw material with PFI will reduce or, improve freshness, and prevent the production of unhealthy and toxic compounds such as biogenic amines like trimethylamine or sulfurous compounds like dimethyl sulfide in the raw material during the waiting period so to improve the freshness, aroma, and quality of rendered meat or seafood meal.

What is claimed is:

1. A method for preparing raw meat or raw seafood that is resistant to spoilage, the method comprising
a) obtaining a solution comprising a population of non-pathogenic psychrotrophic lactic acid bacteria under conditions and for a period of time sufficient to enable growth of the bacterial population to form a bacterial culture capable of preventing spoilage of the raw meat or raw seafood;
b) adding a saccharide to the bacterial culture formed in step a) and incubating the bacterial culture at a temperature of from 1 to 4° C., wherein the saccharide is present in an amount sufficient to achieve bacteriostasis of the bacterial culture;
c) admixing the bacterostatic bacterial culture formed in step b) with the raw meat or raw seafood, wherein the bacteriostatic bacterial culture is present in an amount of from 1 to 20 weight % based on the total weight of the composition, and is capable of preventing spoilage of the raw meat or seafood for at least 6 days at a temperature of from 5 to 9° C.

2. A method according to claim 1, wherein in step c) the bacteriostatic bacterial culture is present in an amount of from 2.5 to 5.0 weight %.

3. A method according to claim 1, wherein in step b) the saccharide is present in an amount of from 50 to 80 weight % based on the total weight of the composition of step b).

4. A method according to claim 2, wherein the saccharide is present in an amount of from 55 to 80 weight %.

5. A method according to claim 1, which method prevents growth of spoilage psychrotrophic bacteria in the raw meat or raw seafood.

6. A method according to claim 1, wherein in step a) the solution comprises one or more nutrients sufficient to enable growth of the bacterial population.

7. A method according to claim 6, wherein the one or more nutrients are selected from the group consisting of saccharides, a nitrogen source, fat, vitamins, minerals, micronutrients and combinations thereof.

8. A method according to claim 7, wherein the nitrogen source is selected from the group consisting of peptides, peptone, protein, hydrolyzed protein, nucleotides, yeast, yeast extract or whey.

9. A method according to claim 7, wherein the minerals and micronutrients are selected from the group consisting of sodium, potassium, calcium, magnesium, phosphate, chloride, and sulfur.

10. A method according to claim 1, wherein in step a) the solution comprises a saccharide in an amount of from 1 to 30 weight %.

11. A method according to claim 1, wherein the solution comprises whey and a saccharide.

12. A method according to claim 11, wherein the saccharide is dextrose.

13. A method according to claim 1, wherein prior to step a) a culture of the non-pathogenic psychrotrophic lactic acid bacteria is added to growth media in an amount of from 0.05 to 2 weight % to form the solution.

14. A method according to claim 1, wherein the non-pathogenic psychrotrophic lactic acid bacteria are selected from the group consisting of Streptococcus, Enterococcus, Lactobacillus, Bifidobacterium, Pediococcus, Leuconotoc, and any combinations thereof.

15. A method according to claim 14, wherein the non-pathogenic psychrotrophic lactic acid bacteria are selected from the group consisting of Streptococcus thermophilus, Lactobacillus delbrueckli subsp. bulgaricus, Lactobacillus acidophilus, Bifidobacterium lactis and combinations thereof.

16. A method according to claim 1, wherein in step a) the solution is incubated at a temperature of from 10° C. to 25° C.

17. A method according to claim 1, wherein in step a) incubation occurs until a total bacterial population of 10⁸ colony forming units per gram of solution is achieved.

18. A method according to claim 1, wherein in step a) the growth of the bacterial population is detected by a decrease in pH of the solution.

19. A method according to claim 1, wherein in step a) the period of time sufficient to enable growth of the bacterial population to form a bacterial culture capable of preventing spoilage of the raw meat or raw seafood is from 24 to 48 hours.

20. A method according to claim 1, wherein the saccharide added in step b) is a monosaccharide or a disaccharide.

21. A method according to claim 20, wherein the saccharide is selected from dextrose, sucrose, lactose, galactose, fructose, xylose, high fructose corn syrup and any combination thereof.

22. A method according to claim 1, wherein prevention of spoilage is measured by one or more of microbial count, change in pH or the identification of an odor compound.

23. A method according to claim 22, wherein spoilage of the raw meat or raw seafood is identified by a pH of from 6.5 or higher.

24. A method according to claim 22, wherein the odor compound is identified by determining the quantity of odor compounds by gas-phase chromatography mass spectrometry.

25. A method according to claim 24, wherein the odor compounds are volatile organic compounds.

26. A method according to claim 22, wherein the odor compound is selected from one or more of ammonia, hydrogen sulfide, methane thiol, trimethyl amine, methyl disulfide, dimethyl trisulfide, dimethyl sulfide, 2-methylbutanal, 3-methylbutanal, 3-methyl butanoic acid, 1-hexanol, pentanal, hexanal, heptanal, 1-penten-3-ol pentylfuran, benzaldehyde, acetic acid, propionic acid, pentanoic acid, hexanoic acid and heptanoic acid.

27. A method according to claim 1, wherein spoilage of the raw meat or raw seafood is prevented for a period of up to 12 days at 6° C.

28. A method according to claim 1, wherein the total aerobic bacterial count of the raw meat or fish is less than or equal to 10⁵ after incubation with the bacterial culture for 8 days.

29. A method according to claim 1, wherein the total aerobic bacterial count of the raw meat or fish is less than or equal to 10⁶ after incubation with the bacterial culture for 12 days.

30. A method according to claim 5, wherein the spoilage psychrotrophic bacteria are selected from the group consisting of pseudomonas, campylobacter, and listeria.

31. A method according to claim 1, wherein the raw meat or raw seafood is from 500 kg to 30000 kg of raw meat or raw seafood.

32. A method according to claim 1, wherein the raw meat or raw seafood is ground meat or ground seafood, or meat slurry or seafood slurry.

33. A method according to claim 1, wherein the raw meat is ground beef or ground chicken.

34. A method according to claim 1, wherein the raw mea or raw seafood is substantially free from chemical preservative.

35. Raw meat or raw seafood obtainable according to the method of claim 1.

36. A food composition comprising the raw meat or raw seafood according to claim 35.

37. A food composition according to claim 36, which is a pet food composition.

38. A fermentation inoculant comprising bacteriostatic non-pathogenic psychrotrophic lactic acid bacteria; and saccharide in an amount of at least 50 weight %.

39. A fermentation inoculant according to claim 38, wherein the non-pathogenic psychrotrophic lactic acid bacteria are selected from the group consisting of Streptococcus, Enterococcus, Lactobacillus, Bifidobacterium, Pediococcus, Leuconotoc, and any combinations thereof.

40. A fermentation inoculant according to claim 39, comprising Streptococcus thermophilus, Lactobacillus delbrueckli subsp. bulgaricus, Lactobacillus acidophilus, and Bifidobacterium lactis.

41. A fermentation inoculant according to claim 38, wherein the saccharide is dextrose.
